SEQUENCE LISTING (8 sequences)

SEQ ID NO: 1 - DNA, 1565 bp, Influenza A virus
agcaaaagcagggtagataatcactcactgagtgacatcaaaatcatggcgtcccaaggcaccaaacggtcttacgaacagatggagactgatggagaacgccagaatgccactgaaatcagagcatccgtcggaaaaatgattggtggaattggacgattctacatccaaatgtgcacagaacttaaactcagtgattatgagggacggttgatccaaaacagcttaacaatagagagaatggtgctctctgcttttgacgaaaggagaaataaatacctggaagaacatcccagtgcggggaaggatcctaagaaaactggaggacctatatacagaagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagggctcttgttcgcaccggaatggatcccaggatgtgctctctgatgcaaggttcaactctccctaggaggtctggagccgcaggtgctgcagtcaaaggagttggaacaatggtgatggaattggtcaggatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccgggacccagggaatgctgagttcgaagatctcacttttctagcacggtctgcactcatattgagagggtcggttgctcacaagtcctgcctgcctgcctgtgtgtatggacctgccgtagccagtgggtacgactttgaaagagagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattgagcttcatcaaagggacgaaggtggtcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccgttatggcagcattcactgggaatacagaggggagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 2 - protein, 498 aa, Influenza A virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRDPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVVPRGKLSTRGVQIASNENMETMESSTLELRSRYWAIRTRSGGNTNQQRASAGQISIQPTFSVQRNLPFDRTTVMAAFTGNTEGRTSDMRTEIIRMMESARPEDVSFQGRGVFELSDEKAASPIVPSFDMSNEGSYFFGDNAEEYDN

SEQ ID NO: 3 - DNA, 1566 bp, Influenza A virus
agcaaaagcagggttaataatcactcaccgagtgacatcaaaatcatggcgtcccaaggcaccaaacggtcttatgaacagatggaaactgatggggatcgccagaatgcaactgagattagggcatccgtcgggaagatgattgatggaattgggagattctacatccaaatgtgcactgaacttaaactcagtgatcatgaagggcggttgatccagaacagcttgacaatagagaaaatggtgctctctgcttttgatgaaagaaggaataaatacctggaagaacaccccagcgcggggaaagatcccaagaaaactggggggcccatatacaggagagtagatggaaaatggatgagggaactcgtcctttatgacaaagaagagataaggcgaatctggcgccaagccaacaatggtgaggatgcgacagctggtctaactcacataatgatctggcattccaatttgaatgatgcaacataccagaggacaagagctcttgttcgaactggaatggatcccagaatgtgctctctgatgcagggctcgactctccctagaaggtccggagctgcaggtgctgcagtcaaaggaatcgggacaatggtgatggaactgatcagaatggtcaaacgggggatcaacgatcgaaatttctggagaggtgagaatgggcggaaaacaagaagtgcttatgagagaatgtgcaacattcttaaaggaaaatttcaaacagctgcacaaagagcaatggtggatcaagtgagagaaagtcggaacccaggaaatgctgagatcgaagatctcatatttttggcaagatctgcattgatattgagagggtcagttgctcacaaatcttgcctacctgcctgtgcgtatggacctgcagtatccagtgggtacgacttcgaaaaagagggatattccttggtgggaatagaccctttcaaactacttcaaaatagccaaatatacagcctaatcagacctaacgagaatccagcacacaagagtcagctggtgtggatggcatgccattctgctgcatttgaagatttaagattgttaagcttcatcagagggacaaaagtatctccgcgggggaaactgtcaactagaggagtacaaattgcttcaaatgagaacatggataatatgggatcgagcactcttgaactgagaagcgggtactgggccataaggaccaggagtggaggaaacactaatcaacagagggcctccgcaggccaaaccagtgtgcaacctacgttttctgtacaaagaaacctcccatttgaaaagtcaaccatcatggcagcattcactggaaatacggagggaaggacttcagacatgagggcagaaatcataagaatgatggaaggtgcaaaaccagaagaagtgtcattccgggggaggggagttttcgagctctcagacgagaaggcaacgaacccgatcgtgccctcttttgatatgagtaatgaaggatcttatttcttcggagacaatgcagaagagtacgacaattaaggaaaaaatacccttgtttctact

SEQ ID NO: 4 - protein, 498 aa, Influenza A virus
MASQGTKRSYEQMETDGDRQNATEIRASVGKMIDGIGRFYIQMCTELKLSDHEGRLIQNSLTIEKMVLSAFDERRNKYLEEHPSAGKDPKKTGGPIYRRVDGKWMRELVLYDKEEIRRIWRQANNGEDATAGLTHIMIWHSNLNDATYQRTRALVRTGMDPRMCSLMQGSTLPRRSGAAGAAVKGIGTMVMELIRMVKRGINDRNFWRGENGRKTRSAYERMCNILKGKFQTAAQRAMVDQVRESRNPGNAEIEDLIFLARSALILRGSVAHKSCLPACAYGPAVSSGYDFEKEGYSLVGIDPFKLLQNSQIYSLIRPNENPAHKSQLVWMACHSAAFEDLRLLSFIRGTKVSPRGKLSTRGVQIASNENMDNMGSSTLELRSGYWAIRTRSGGNTNQQRASAGQTSVQPTFSVQRNLPFEKSTIMAAFTGNTEGRTSDMRAEIIRMMEGAKPEEVSFRGRGVFELSDEKATNPIVPSFDMSNEGSYFFGDNAEEYDN

SEQ ID NO: 5 - DNA, 1565 bp, Influenza A virus
agcaaaagcagggtagataatcactcaccgagtgacatcaacatcatggcgtctcaaggcaccaaacgatcttatgaacagatggaaactggtggagaacgccagaatgctactgagattagggcatctgttggaagaatggttagtggcattgggaggttctacatacagatgtgcacagaactcaaactcagtgactatgaagggaggctgatccagaacagcataacaatagagagaatggtgctctctgcatttgatgaaagaaggaacagatacctggaagaacaccccagtgcggggaaggacccgaagaaaactggaggtccaatttatcggaggagagacgggaaatgggtgagagagctgattctgtatgacaaagaggagatcaggaggatttggcgtcaagcgaacaatggagaggacgcgactgctggtcttacccacctgatgatatggcattccaacctaaatgatgccacatatcagagaacgagagctctcgtgcgtactggaatggatcccaggatgtgctctctgatgcaaggatcaactctcccgaggagatctggagctgccggtgcagcagtaaagggggtagggacaatggtgatggagctgattcggatgataaagcgagggatcaacgaccggaatttctggagaggtgaaaatggaagaagaacaaggattgcatatgagagaatgtgtaacatcctcaaagggaaattccaaacagcagcacaaagagcaatgatggatcaagtgcgagagagcagaaatcctgggaatgctgaaattgaagatctcatttttctggcacggtctgcactcatcctgagaggatcagtggcccataaatcctgcttgcctgcttgtgtgtacggacttgcagtggccagtggatatgactttgagagagaagggtactctctggttggaatagatcctttccgtctgcttcaaaacagccaggtctttagtctcattagaccaaatgagaatccagcacataagagtcaattagtgtggatggcatgccactctgcagcatttgaggaccttagagtctcaagtttcatcagagggacaagagtggtcccaagaggacagctatccaccagaggggttcaaattgcctcaaatgagaacatggaagcaatggactccaacactcttgaactgagaagtagatattgggctataagaaccagaagcggaggaaacaccaaccagcggagggcatctgcaggacagatcagcgttcagcccactttctcagtacagagaaatcttcccttcgaaagagcaaccattatggcagcatttacagggaatactgagggcagaacgtctgacatgaggactgaaatcataggaatgatggaaagtgccagaccagaagatgtgtcattccaggggcggggagtcttcgagctctcggacgaaaaggcaacgaacccgatcgtgccttcctttgacatgaataatgaaggatcttatttcttcggagacaatgcagaggagtatgacaattaaagaaaaatacccttgtttctact

SEQ ID NO: 6 - protein, 498 aa, Influenza A virus
MASQGTKRSYEQMETGGERQNATEIRASVGRMVSGIGRFYIQMCTELKLSDYEGRLIQNSITIERMVLSAFDERRNRYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMEAMDSNTLELRSRYWAIRTRSGGNTNQRRASAGQISVQPTFSVQRNLPFERATIMAAFTGNTEGRTSDMRTEIIGMMESARPEDVSFQGRGVFELSDEKATNPIVPSFDMNNEGSYFFGDNAEEYDN

SEQ ID NO: 7 - DNA, 1515 bp, Influenza A virus
tgagtgacatcaacatcatggcgtctcaaggcaccaaacgatcttatgaacagatggaaactggtggagaacgccagaatgccactgagatcagggcatccgttggaagaatggttggtggaattgggaggttttacatacagatgtgcactgaactcaaactcagcgaccaggaaggaaggttgatccagaacagtataacagtagagagaatggttctctctgcatttgatgaaaggaggaacaggtacctagaggaacatcccagtgcggggaaggacccgaagaagaccggaggtccaatctaccgaagaagaaacgggaaatgggtgagagagctgattctgtatgacaaagaggagataaggagaatttggcgccaagcgaacaatggagaagacgcaactgctggtctcactcacatgatgatttggcattccaacctaaatgatgccacataccagagaacaagagccctcgtgcggactggaatggaccccagaatgtgctctctgatgcaaggatcaaccctcccgaggagatctggagctgctggtgcagcaataaagggagtcgggacgatggtaatggaactaattcggatgataaagcgaggcattaatgaccggaacttctggagaggcgagaatggacgaagaacaaggattgcatatgagagaatgtgcaacatcctcaaagggaaatttcaaacagcagcacaaaaagcaatgatggatcaggtgcgagaaagcagaaatcctgggaatgctgaaattgaagatctcatttttctggcacggtctgcactcatcctgagaggatccgtagcccataagtcctgcttgcctgcttgtgtgtacggactcgctgtggccagtggatatgattttgagagggaagggtactctctggttgggatagatcctttccgtctgcttcagaacagtcaggtcttcagtctcattagaccaaaagagaatccagcacataaaagtcaattggtatggatggcatgccattctgcagcatttgaggacctgagagtctcaagtttcattagaggaacaagagtaatcccaagaggacaactatccactagaggagttcagattgcttcaaatgagaacgtggaagcaatggactccagcactcttgaactgagaagcagatattgggctataaggaccaggagtggaggaaacaccaaccaacagagagcatctgcaggacaaatcagtgtacagcccactttctcagtacagagaaatcttcccttcgaaagagtgaccattatggccgcgtttaaggggaataccgagggcagaacatctgacatgaggactgaaatcataagaatgatggaaagtgccagaccagaagatgtgtctttccaggggcggggagtcttcgagctctcagacgaaaaggcaacgaacccgatcgtgccttcctttgacatgagtaatgaaggatcttatttcttcggagacaatgcagaggaatatgacaattgaa

SEQ ID NO: 8 - protein, 498 aa, Influenza A virus
MASQGTKRSYEQMETGGERQNATEIRASVGRMVGGIGRFYIQMCTELKLSDQEGRLIQNSITVERMVLSAFDERRNRYLEEHPSAGKDPKKTGGPIYRRRNGKWVRELILYDKEEIRRIWRQANNGEDATAGLTHMMIWHSNLNDATYQRTRALVRTGMDPRMCSLMQGSTLPRRSGAAGAAIKGVGTMVMELIRMIKRGINDRNFWRGENGRRTRIAYERMCNILKGKFQTAAQKAMMDQVRESRNPGNAEIEDLIFLARSALILRGSVAHKSCLPACVYGLAVASGYDFEREGYSLVGIDPFRLLQNSQVFSLIRPKENPAHKSQLVWMACHSAAFEDLRVSSFIRGTRVIPRGQLSTRGVQIASNENVEAMDSSTLELRSRYWAIRTRSGGNTNQQRASAGQISVQPTFSVQRNLPFERVTIMAAFKGNTEGRTSDMRTEIIRMMESARPEDVSFQGRGVFELSDEKATNPIVPSFDMSNEGSYFFGDNAEEYDN

(19) United States
(12) Patent Application Publication
(10) Pub. No.: US 2012/0142701 A1
(43) Pub. Date: Jun. 7, 2012

(54) COMPOUNDS AND METHODS FOR THE TREATMENT OF PROLIFERATIVE DISEASES

(75) Inventors: Yi Tsun Richard Kao, Hong Kong (CN); Kwok-Yung Yuen, Hong Kong (CN)

(73) Assignee: The University of Hong Kong

(21) Appl. No.: 13/149,056

(22) Filed: May 31, 2011

Related U.S. Application Data
(60) Provisional application No. 61/349,565, filed on May 28, 2010.
(60) Provisional application No. 61/349,525, filed on May 28, 2010.

(51) Int. Cl. A61K 31/496; A61P 31/12; A61P 31/04; G01N 33/566; A61P 31/16; G01N 33/53; C12Q 1/68; C07D 417/06; A61P 35/00

(52) U.S. Cl. 514/254.04; 544/367; 435/7.1; 435/6.19; 436/501

(57) ABSTRACT

Cell-based and cell-free assays are disclosed that detect compounds that promote aggregation of proteins, glycoproteins, and protein-nucleic acid complexes. Also disclosed are pharmaceutical formulations useful for treating or preventing viral infections, bacterial infections, cancer, and diseases involving hyper-proliferative cells.

### CROSS-REFERENCE TO RELATED APPLICATIONS

This application claims priority to U.S. Ser. No. 61/349,565 entitled “Compounds and Methods for the Treatment of Proliferative Diseases”, filed May 28, 2010, and U.S. Ser. No. 61/349,525 entitled “Compounds and Methods for the Treatment of Viral Infections”, filed May 28, 2010, the contents of each being incorporated herein by reference.

### FIELD OF THE INVENTION

The present invention relates to methods of identifying compounds that may be useful for the treatment or prevention of proliferative diseases, in particular compounds which promote protein aggregation, and methods of making and using thereof.

### REFERENCE TO SEQUENCE LISTING

The Sequence Listing submitted May 31, 2011 as a text file named “UHK_{—}00359_ST25.txt,” created on May 31, 2011, and having a size of 26,192 bytes is hereby incorporated by reference pursuant to 37 C.F.R. §1.52(e)(5).

### BACKGROUND OF THE INVENTION

Aggregation of proteins has been suggested as a cause or result of a number of diseases (Nature Reviews Drug Discovery. 2010; 9: 237-248) and a significant hurdle in drug development (Int J Pharm. 2005; 289: 1-30). Traditionally, compounds that cause aggregation of proteins are usually excluded from further development due to a fear of adverse effects when administered to patients. However, identifying agents that inhibit protein aggregation, such as amyloid aggregation in Alzheimer's disease patients, has been the subject of concerted research efforts.

While many scientific efforts focus on preventing aggregation, compounds that promote aggregation of proteins, glycoproteins, and protein-nucleic acid complexes can be used as therapeutics. Aggregation of proteins, glycoproteins, and protein-nucleic acid complexes provides a mechanism for abolishing the replication of the organism. For example, in viral infections, the viral nucleoprotein must enter the nucleus of the host cell to undergo transcription. Aggregation outside of the nucleus, such as in the cytosol, is one method of preventing the migration of viral nucleoprotein. Compounds which promote such aggregation could potentially be useful in both understanding the mechanism(s) of protein aggregation in vitro and in vivo and elucidating the role of aggregation in a number of disease states. Furthermore, compounds which promote aggregation may be useful in treating disease of hyper-proliferation, such as cancer or various infective diseases.

There is a need for methods of identifying compounds which promote aggregation of proteins, glycoproteins, and protein-nucleic acid complexes in vitro and in vivo as a method of preventing diseases. There is also a need for anti-bacterial, anti-viral, anti-cancer, and anti-proliferative formulations containing these compounds that treat and/or prevent the spread of unwanted cells or infections.

Therefore, it is an object of the present invention to provide assays for identifying compounds that promote aggregation, in particular compounds which interfere with the biological activities of proteins, glycoproteins, and protein-nucleic acid complexes.

It is a further object of the invention to provide methods of making and using small molecules that promote aggregation.

It is yet further an object of the invention to provide pharmaceutical compositions and formulations that effectively treat or prevent bacterial infections, viral infections, cancer, and/or hyper-proliferative diseases, for example, by providing a therapeutically effective amount of the compound to promote protein aggregation.

It is an object of the invention to provide uses of the compounds in the manufacture of a medicament for the treatment or for the prevention of bacterial infections, viral infections, cancer, and/or hyper-proliferative diseases.

It is another object of the invention to provide uses of the compositions or formulations in the manufacture of a medicament for the treatment or for the prevention of bacterial infections, viral infections, cancer, and/or hyper-proliferative diseases.

### SUMMARY OF THE INVENTION

Cell-based and cell-free assays have been developed to identify compounds that promote cytoplasmic nucleoprotein aggregation and inhibit nuclear accumulation of nucleoprotein. Also disclosed are compounds according to formula I:

Ar¹—Y—Ar²—X—Cy—Z—Ar³ (Formula I)

wherein, Ar¹, Ar², and Ar³ are each independently substituted or unsubstituted aryl or heteroaryl groups;

X, Y, and Z are independently absent (i.e, a direct bond) or selected from —C(═O)—, —S(═O)—, —SO₂—, —C(═O)N(R₁), —N(R₂)—, —C(R₃)═C(R₄)—, and —C(R₅R₆)ₙ;

n is 0 to 10, preferably 0-6;

R₁-R₆ are each independently selected from hydrogen; halogen; hydroxy; nitro; nitrile; isonitrile; urea; guanidine; cyano; carbonyl, such as formyl, acyl, or carboxyl; thiocarbonyl, such as thioester, thioacetate, or thioformate; primary, secondary, or tertiary amine (i.e., amino); amide; amidine; imine; azide; thiol, substituted or unsubstituted thioalkyl (e.g., thioether); isocyanate; isothiocyanate; phosphoryl; phosphate; phosphinate; sulfate; sulfonate; sulfamoyl; sulfonamide; sulfonyl; substituted or unsubstituted linear or branched alkyl, substituted or unsubstituted linear or branched alkenyl, substituted or unsubstituted linear or branched alkynyl, substituted or unsubstituted linear and branched alkoxy, substituted or unsubstituted C₃-C₁₀ cycloalkyl, cycloalkenyl, heterocyloalkyl, or heterocycloalkenyl, substituted or unsubstituted aryl or heteroaryl; and

Cy is a 5-7 membered substituted or unsubstituted cyclic or heterocyclic group.

Methods of treating and/or preventing viral infections, bacterial infections, cancer, and/or hyper-proliferative diseases by administering a compound that promotes aggregation are also described herein. In a preferred embodiment, compounds and/or formulations are used to treat influenza infection, in particular influenza A infections. Preferred influenza strains to be treated include H1N1, H3N2, and H5N1. In a preferred embodiment, the compositions are part of a formulation that can be administered orally or parenterally to a patient in need thereof. In a particularly preferred embodiment, the compositions are administered orally.

### BRIEF DESCRIPTION OF THE DRAWINGS

FIG. 1 shows a dose-response curve for nucleozin-treated mammalian cells infected with influenza A H1N1, H3N2, and H5N1 strains.

FIG. 2 shows a survival curve for nucleozin-treated (filled square) or untreated mice (open triangle) when challenged with the highly pathogenic A/Vietnam/1194/04 H5N1 virus.

FIG. 3 is a plot of the antiviral activity of an aggregation-inducing agent nucleozin in multicycle growth assays. Madin-Darby Canine Kidney (MDCK) cells were infected with A/WSN/33 virus at 0.001 MOI in the presence or absence of nucleozin (0.1 or 1 μM). Viral titres were determined by plaques assay at the time indicated. Nucleozin suppressed viral growth at 0.1 μM and completely inhibited virus production at 1

FIG. 4 is a graph showing the time-dependent (seconds) nucleozin induced aggregation of nucleoprotein (radius).

### DETAILED DESCRIPTION OF THE INVENTION

### I. Definitions

“Aggregation” as generally used herein refers to the consolidation of proteins, glycoproteins, or protein-nucleic acid complexes inside a cell, such that upon observation with an imaging technique, such as fluorescence microscopy, dense aggregates of proteins, glycoproteins, or protein-nucleic acid complexes are visible. Aggregation can be visualized as a halo of dense material inside the cell, preferably outside the nucleus. Preferably, aggregation occurs as a result of treatment with a compound which binds a protein, glycoprotein, or protein-nucleic acid complex. Binding or complexing may involve covalent or non-covalent interactions, weak to strong intermolecular forces, including, but not limited to, covalent bonds, hydrogen bonds, disulfide bonds, salt bridges, ionic bonds, metal coordination, hydrophobic forces, van der Waals interactions, cation-pi interactions, pi-stacking, and combinations thereof. Aggregation typically results in the inability of proteins, glycoproteins, or protein-nucleic acid complexes to carry out biological functions.

“Anti-proliferative” as generally used herein refers to compounds which prevent cellular growth or viral replication when administered to cells.

“Alkyl” as generally used herein refers to the radical of saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl generally has 30 or fewer carbon atoms in its backbone (e.g., C₁-C₃₀ for straight chain, C₃-C₃₀ for branched chain), preferably 20 or fewer, preferably 10 or fewer, more preferably 6 or fewer, most preferably 5 or fewer. If the alkyl is unsaturated, the alkyl chain generally has from 2-30 carbons in the chain, preferably from 2-20 carbons in the chain, preferably from 2-10 carbons in the chain, more preferably from 2-6 carbons, most preferably from 2-5 carbons. Likewise, preferred cycloalkyls have from 3-20 carbon atoms in their ring structure, preferably from 3-10, more preferably from 3-6 carbon atoms in their ring structure, most preferably 5, 6 or 7 carbons in the ring structure. Examples of saturated hydrocarbon radicals include, but are not limited to, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, and 3-butynyl.

The term “alkyl” includes one or more substitutions at one or more carbon atoms of the hydrocarbon radical as well as heteroalkyls. Suitable substituents include, but are not limited to, halogens, such as fluorine, chlorine, bromine, or iodine; hydroxyl; —NR₁R₂, wherein R₁ and R₂ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CONR₂, wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, —CF₃; —CN; —NCOCOCH₂CH₂; —NCOCOCHCH; —NCS; and combinations thereof

“Aryl,” as generally used herein, refers to a carbon based aromatic ring having 3-20, preferably 5-15, more preferably 6-10 ring members, including phenyl, biphenyl, or naphthyl. The aryl group can be optionally substituted with one or more moieties selected from the group consisting of hydroxyl, acyl, amino, halo, alkylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al. Protective Groups in Organic Synthesis, John Wiley and Sons, Third Edition, 2002. The term “aryl” includes one or more substitutions at one or more carbon atoms of the hydrocarbon radical. Suitable substituents include, but are not limited to, halogens, such as fluorine, chlorine, bromine, or iodine; hydroxyl; —NR₁R₂, wherein R₁ and R₂ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CONR₂, wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, —CF₃; —CN; —NCOCOCH₂CH₂; —NCOCOCHCH; —NCS; and combinations thereof.

“Effective amount” as generally used herein refers to an amount, or dose, within the range normally given or prescribed to demonstrate an effect, e.g., in vitro or in vivo. The range of an effective amount may vary from individual to individual; however, the optimal dose is readily determinable by those of skill in the art depending upon the use. Such ranges are well established in routine clinical practice and will thus be readily determinable to those of skill in the art. Doses may be measured by total amount given (e.g. per dose or per day) or by concentration. Doses of 0.01, 0.05, 0.1, 0.5, 1, 2, 3, 4, 5, 6, 7, 8, 9, 10, 15, 20, 25, 30, 40, 50, 100, 500 and 1000 mg/kg/day may be appropriate for treatment.

“Heterocycle” or “heterocyclic” as generally used herein refers to one or more rings of 5-12 atoms, preferably 5-7 atoms, with or without unsaturation or aromatic character and having at least one ring atom which is not a carbon. Preferred heteroatoms include sulfur, oxygen, and nitrogen. Multiple rings may be fused, as in quinoline or benzofuran. Particularly preferred heterocycle groups are 5-10-membered rings with 1-3 heteroatoms selected from O, S, P, Si, As, and N. Heterocycles include, but are not limited to azolidine, pyrrole, oxolane, furan, thiolane, thiophene, phospholane, phosphole, silane, silole, arsolane, arsole, imidazoline, pyrazolidine, imidazole, imidazoline, pyrazole, pyrazoline, oxazolidine, isoxazolidine, oxazole, oxazoline, isoxazole, isoxazoline, thiazolidine, isothiazolidine, thiazole, thiazoline, isothiazole, isothiazoline, dioxolane, oxathiolane, dithiolane, thiazole, dithiazole, furazan, oxadiazole, thiadiazole, tetrazole, piperidine, pyridine, pyran, tetrahydropyran, thiane, thiopyran, piperazine, diazine, morpholine, oxazine, thiazine, dithiane, dioxane, dioxin, triazine, trioxane, tetrazine, azapane, azepine, oxepane, oxepine, thiepane, thiepine, azocane, azocine, oxecane, and thiocane. Heterocycle or heterocyclic also refers to substituted rings, as defined in “aryl” or “alkyl.”

The term “heterocycle” includes one or more substitutions at one or more carbon or heteroatoms. Suitable substituents include, but are not limited to, halogens, such as fluorine, chlorine, bromine, or iodine; hydroxyl; —NR₁R₂, wherein R₁ and R₂ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CONR₂, wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, —CF₃; —CN; —NCOCOCH₂CH₂; —NCOCOCHCH; —NCS; and combinations thereof.

“Heteroaryl” as generally used herein refers to an aromatic group having 3-20, preferably 5-14, more preferably 6-10 ring members and containing from one to four N, O, P, Si, As, or S atoms(s) or a combination thereof, which heteroaryl group is optionally substituted at carbon or nitrogen atom(s). Heteroaryl rings may also be fused with one or more cyclic hydrocarbon, heterocyclic, aryl, or heteroaryl rings. Heteroaryl includes, but is not limited to, 5-membered heteroaryls having one hetero atom (e.g., thiophenes, pyrroles, furans); 5 membered heteroaryls having two heteroatoms in 1,2 or 1,3 positions (e.g., oxazoles, pyrazoles, imidazoles, thiazoles, purines); 5-membered heteroaryls having three heteroatoms (e.g., triazoles, thiadiazoles); 5-membered heteroaryls having 3 heteroatoms; 6-membered heteroaryls with one heteroatom (e.g., pyridine, quinoline, isoquinoline, phenanthrine, 5,6-cycloheptenopyridine); 6-membered heteroaryls with two heteroatoms (e.g., pyridazines, cinnolines, phthalazines, pyrazines, pyrimidines, quinazolines); 6-membered heteroaryls with three heteroatoms (e.g., 1,3,5-triazine); and 6-membered heteroaryls with four heteroatoms. Particularly preferred heteroaryl groups are 5-10-membered rings with 1-3 heteroatoms selected from O, S, and N.

The term “heteroaryl” includes one or more substitutions at one or more carbon or heteroatoms atoms. Suitable substituents include, but are not limited to, halogens, such as fluorine, chlorine, bromine, or iodine; hydroxyl; —NR₁R₂, wherein R₁ and R₂ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CONR₂, wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, —CF₃; —CN; —NCOCOCH₂CH₂; —NCOCOCHCH; —NCS; and combinations thereof.

“Substituted”, as used herein, means one or more positions on the functional group are substituted with one or more groups including, but not limited to; halogen (e.g., fluorine, chlorine, bromine, and iodine); hydroxy; nitro; nitrile; isonitrile; urea; guanidine; cyano; carbonyl, such as formyl, acyl, or carboxyl; thiocarbonyl, such as thioester, thioacetate, or thioformate; primary, secondary, tertiary, or quaternary amine (i.e., amino); amide; amidine; imine; azide; thiol, substituted or unsubstituted thioalkyl (e.g., thioether); isocyanate; isothiocyanate; phosphoryl; phosphate; phosphinate; sulfate; sulfonate; sulfamoyl; sulfonamide; sulfonyl; alkyl, alkenyl, alkynyl, alkoxy, cycloalkyl, cycloalkenyl, heterocyloalkyl, or heterocycloalkenyl, aryl or heteroaryl.

“Hits” as generally used herein refers to a compound which shows the desired activity or potency in a screening assay.

“Influenza A” as generally used herein refers to mammalian Influenza A virus, e.g., H3N2, H1N1, H2N2, H7N7 and H5N1 (avian influenza virus) strains and variants thereof.

“Nucleoprotein” or “NP” as generally used herein refers to any protein that is structurally associated with nucleic acid. Exemplary nucleoproteins are identified and sequenced in certain strains of influenza viruses. Exemplary sequences can be found in the NCBI database. The GenBank accession numbers of some exemplary NP sequences from influenza type A for subtype H1N1 are NP 040982 (AAA43467) (SEQ ID NO: 1 AND SEQ ID NO: 2), for subtype H3N2 are AAZ38620 (YP308843) (SEQ ID NO: 3 AND SEQ ID NO: 4); and for subtype H5N1 are AY856864 (SEQ ID NO: 5 AND SEQ ID NO: 6) and AAF02400 (SEQ ID NO: 7 AND SEQ ID NO: 8).

“Nucleozin” as generally referred to herein, refers to an exemplary nucleoprotein inhibitor which both inhibits nuclear nucleoprotein accumulation and promotes nucleoprotein aggregation. Nucleozin has the chemical structure as follows:

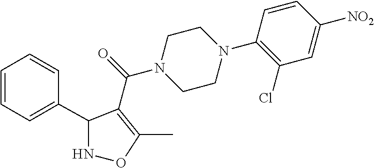

“Pharmaceutically acceptable” as generally used herein refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Pharmaceutically acceptable salts” as generally used herein refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic.

“Substituted” as generally used herein refers to a moiety (e.g., an alkyl group or aryl group) substituted with one or more substituents including, but not limited to: halogen; hydroxy; nitro; nitrile; isonitrile; urea; guanidine; cyano; carbonyl, such as formyl, acyl, or carboxyl; thiocarbonyl, such as thioester, thioacetate, or thioformate; primary, secondary, or tertiary amine (i.e., amino); amide; amidine; imine; azide; thiol, substituted or unsubstituted thioalkyl (e.g., thioether); isocyanate; isothiocyanate; phosphoryl; phosphate; phosphinate; sulfate; sulfonate; sulfamoyl; sulfonamide; sulfonyl; substituted or unsubstituted linear or branched alkyl, alkenyl, or alkynyl; substituted or unsubstituted linear or branched alkoxy; substituted or unsubstituted C₃-C₁₀ cycloalkyl, cycloalkenyl, heterocyloalkyl, or heterocycloalkenyl; substituted or unsubstituted aryl or heteroaryl.

“Substituted aryl” as generally used herein refers to aryl groups having one or more non-interfering groups as a substituent. For substitutions on a phenyl ring, the substituents may be in any orientation (i.e., ortho, meta, or para).

“Test compound(s)” as generally used herein refers to new or known small molecules (or libraries of molecules) subjected to the one or more assays described herein.

### II. Methods for Identifying Compounds that Promote Aggregation

Compounds which bind to a nuclear protein can promote a conformational change in the nucleoprotein complex, thereby disabling the complex from entering the nucleus. Consequently, the nucleoprotein aggregates in the cytosolic area around the nucleus. Methods of detecting compounds which inhibit nuclear nucleoprotein accumulation and promote nucleoprotein aggregation are disclosed below.

Screening assays to identify agents that interfere with nucleoprotein accumulation or promote cytosolic nucleoprotein aggregation can be used to identify compounds isolated from natural sources such as plants, animals or even sources such as marine, forest or soil samples. It will be understood that the pharmaceutical agents to be screened could also be derived from chemical compositions or man-made compounds.

Test compounds may be found and/or isolated from a variety of custom and commercially available combinatorial libraries. The compounds may be used in combination as required. Moreover, the compounds may be used either in the free form or, if capable of forming salts, in the form of a salt with a suitable acid or base.

In some embodiments, the methods described herein are used to identify possible compounds as anti-proliferative agents including anti-bacterial, anti-cancer, and anti-viral compounds. In a preferred embodiment, the nucleoprotein is the influenza A nucleoprotein and the compounds identified by the methods are hit compounds and potential anti-viral agents.

A. Cell-Based Assay

A cell-based method for identifying compounds that promote aggregation includes:

a.) treating cells with one or more test compounds for a period of time;

b.) adding a fixing solution to stop protein translation;

c.) treating the cells with a fluorescent antibody that binds specifically to a protein, glycoprotein, or protein-nucleic acid complex;

d.) determining the presence or absence of aggregation,

wherein if aggregation is present in (d), the test compound is identified as a compound that may promote aggregation.

In one embodiment, the assay is done in a multi-well format.

In some embodiments, cells are treated with test compound for 24 hours, preferably 12 hours, more preferably 6 hours, most preferably 3 hours.

Techniques for visualizing protein aggregation are well known to those skilled in the art. In the preferred embodiment, the presence or absence of a “halo” of nucleoprotein material in the cytosol is used as the criterion for determining protein aggregation. This is traditionally done by immunofluorescence microscopy.

In some embodiments, washes or aspirations can be done between steps.

In some embodiments, mammalian cell lines such as A549, MDCK, Vero, human fibroblast, or human macrophages can be used. Other cell lines well known to those skilled in the art can also be used.

A non-limiting exemplary procedure is included below:

1. 25 μl of culture medium is added into each well of a 384-well plate followed by the addition of a test compound from a chemical library in each well. Then 25 μl of cells (6×10³ cells/well) with undesirable physiological conditions in DMEM with 10% fetal bovine serum are seeded into each assay well. Plates are incubated at 37° C. in a 5% CO₂ humid atmosphere for 1 day.

2. The supernatant is removed from each well, and washed with 50 μl of phosphate-buffered saline (PBS).

3. The PBS is aspirated off and 50 μl of a cold (4° C.) fixing solution of 3.65% formaldehyde in PBS was added to each well. The plate was incubated for 1 h at 4° C.

4. The fixing solution was aspirated off and 50 μl of 0.1% Nonidet P-40 solution in PBS was added to each well. The plate was incubated for 15 min at room temperature.

5. The Nonidet P-40 solution is aspirated off and each well is blocked with 100 μl 3% milk in PBS for 15 min at room temperature.

6. 25 μl of solution containing fluorescent antibody specific for the proteins, glycoproteins, or protein-nucleic acid complexes is added to each well in 3% milk/PBS. The plates are incubated overnight at 4° C.

7. The antibody solution is aspirated off and the plate was washed twice with 100 μl of 0.05% Tween 20/PBS, 10 min/time.

8. The aggregation of proteins, glycoproteins, or protein-nucleic acid complexes is detected by immunofluorescence microscopy.

In one non-limiting example, nucleozin was used as test compound in the above cell-based assay, and aggregation of nucleoproteins was observed.

B. Cell-Free Assay

A cell-free method for identifying compounds that promote aggregation of proteins, glycoproteins, or protein-nucleic acid complexes includes:

a.) combining a mixture of bovine serum albumin and one or more proteins, glycoproteins, or protein-nucleic acid complexes;

b.) adding the mixture of (a) to a multi-well plate;

c.) transferring a test compound to one or more wells of the multi-well plate;

d.) incubating the plate of (c); and

e.) determining the presence of aggregation,

wherein if aggregation is present in (e), the test compound is identified as a compound that may promote aggregation.

In one embodiment, the assay is done in a multi-well format.

In some embodiments, the plate is incubated for 10 seconds.

In some embodiments, the plate is incubated for 96 hours. In other embodiments, the plate is incubated or any time between 10 seconds to 96 hours

In another embodiment, a plate reader is used to determine aggregation.

In yet another embodiment, centrifugation is used to detect aggregation.

A non-limiting exemplary procedure is shown below:

1. A mixture of 1 μM proteins, glycoproteins, or protein-nucleic acid complexes in 40 μg/ml bovine serum albumin is prepared.

2. A portion (20 μl) of the reaction mixture is added to each well of a 384-well mircotitre plate using automated liquid dispenser.

3. Chemicals from a chemical library are assayed by transferring approximately 100 nl to each assay well by a 384 solid pin array.

4. The plates are incubated at 37° C. for 6 hours and the extent of chemical induced aggregation is recorded by an aggregation plate reader.

In one non-limiting example, nucleozin was used as test compound in the above cell-free assay, and aggregation of nucleoproteins was observed.

### III. Formulations of Aggregation Promoters

A. Compounds

In some embodiments, the compounds have the formulae I-VI below, or pharmaceutically acceptable salts thereof.

In preferred embodiments, the aggregation promoters have the structure of formula I:

Ar¹—Y—Ar²—X—Cy—Z—Ar³ (Formula I)

wherein, Ar¹, Ar², and Ar³ are each independently substituted or unsubstituted aryl or heteroaryl groups;

X, Y, and Z are independently absent (i.e, a direct bond) or selected from —C(═O)—, —S(═O)—, —SO₂—, —C(═O)N(R₁), —N(R₂)—, —C(R₃)═C(R₄)—, and —C(R₅R₆)ₙ—;

n is 0 to 10, preferably 0 to 6; and

R₁-R₆ are each independently selected from hydrogen; halogen; hydroxy; nitro; nitrile; isonitrile; urea; guanidine; cyano; carbonyl, such as formyl, acyl, or carboxyl; thiocarbonyl, such as thioester, thioacetate, or thioformate; primary, secondary, or tertiary amine (i.e., amino); amide; amidine; imine; azide; thiol, substituted or unsubstituted thioalkyl (e.g., thioether); isocyanate; isothiocyanate; phosphoryl; phosphate; phosphinate; sulfate; sulfonate; sulfamoyl; sulfonamide; sulfonyl; substituted or unsubstituted linear or branched alkyl, alkenyl, or alkynyl; substituted or unsubstituted linear or branched alkoxy; substituted or unsubstituted C₃-C₁₀ cycloalkyl, cycloalkenyl, heterocyloalkyl, or heterocycloalkenyl; substituted or unsubstituted aryl or heteroaryl; and

Cy is a 5-7 membered substituted or unsubstituted cyclic or heterocyclic group.

In some embodiments, Ar¹ is substituted with hydrogen, hydroxyl, nitro, amino, or azide; Ar² is substituted with a methyl group; X is C═O; Y and Z are absent; Cy is piperazine; and Ar³ is substituted with a halo group, a nitro group, or a combination of a halo and nitro group.

In some embodiments, Cy is a substituted 5-7 membered unsaturated ring containing 2 nitrogen atoms, wherein one nitrogen atom is bonded to X and another nitrogen atom is bonded to Z.

In a preferred embodiment, Cy is a substituted piperazine, wherein one nitrogen is bonded to X and the second nitrogen is bonded to Z.

In some embodiments, the aggregation promoters have the structure of formula II:

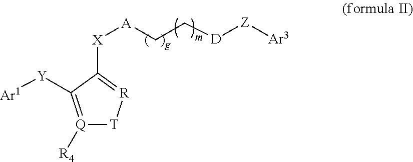

wherein Ar¹ and Ar³ are each independently substituted or unsubstituted aryl or heteroaryl groups;

X, Y, and Z are independently absent or selected from the group consisting of —C(═O)—, —S(═O)—, —SO₂—, —C(═O)N(R₁₀), —N(R₁₁)—, —C(R₁₂)═C(R₁₃)—, and —C(R₁₄R₁₅)ₙ—,

n, g, and m are independently 0 to 10, preferably 0 to 6;

T, Q, and R are, as valence and stability permit, independently selected from C(R₈R₉), nitrogen, oxygen, phosphorous, sulfur, selenium, boron, and arsenic;

A and D are each independently CR₁₆R₁₇ or NR₁₈;

wherein R₄ and R₈-R₁₈ independently are absent, or selected from hydrogen; halogen; hydroxy; nitro; nitrile; isonitrile; urea; guanidine; cyano; carbonyl, such as formyl, acyl, or carboxyl; thiocarbonyl, such as thioester, thioacetate, or thioformate; primary, secondary, or tertiary amine (i.e., amino); amide; amidine; imine; azide; thiol, substituted or unsubstituted thioalkyl (e.g., thioether); isocyanate; isothiocyanate; phosphoryl; phosphate; phosphinate; sulfate; sulfonate; sulfamoyl; sulfonamide; sulfonyl; substituted or unsubstituted linear or branched alkyl, substituted or unsubstituted linear or branched alkenyl, substituted or unsubstituted linear or branched alkynyl, substituted or unsubstituted linear and branched alkoxy, substituted or unsubstituted C₃-C₁₀ cycloalkyl, cycloalkenyl, heterocyloalkyl, or heterocycloalkenyl, substituted or unsubstituted aryl or heteroaryl; or

—CR₁₆R₁₇—, —NR₁₈—, or combinations thereof, when taken together with the optional bridging methylene groups, form a 5-8-membered cyclic structure.

In some embodiments, Ar¹ substituted with hydrogen, hydroxyl, nitro, amino, or azide; X is —C═O; Y and Z are absent, and Ar³ is substituted with a halo group, a nitro group, or a combination of a halo and nitro group. In some embodiment, Ar¹ and A³ are phenyl rings and substituted as described above.

In a preferred embodiment, R₄ is methyl.

In some embodiments, Q is carbon, T is oxygen, and R is nitrogen.

In some embodiments, g and m are 1 and A and D are NR₁₇, wherein A-D defines a piperazine.

In some embodiments, the aggregation promoters have the structure of formula III:

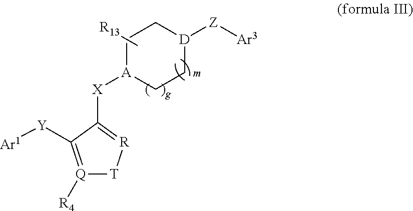

wherein Ar¹ and Ar³ are each independently substituted or unsubstituted aryl or heteroaryl groups;

X, Y, and Z are independently absent or selected from the group consisting of —C(═O)—, —S(═O)—, —SO₂—, —C(═O)N(R₁₀), —N(R₁₁)—, —C(R₁₂)═C(R₁₃)—, and —C(R₁₄R₁₅)ₙ—,

n, g, and m are independently 0 to 10, preferably 0-6;

A, D, T, Q, and R are, as valence and stability permit, independently selected from C(R₈R₉), nitrogen, oxygen, phosphorous, silicon, sulfur, selenium, boron and arsenic;

wherein R₄ and R₈-R₁₅ independently are absent, or are selected from hydrogen; halogen; hydroxy; nitro; nitrile; isonitrile; urea; guanidine; cyano; carbonyl, such as formyl, acyl, or carboxyl; thiocarbonyl, such as thioester, thioacetate, or thioformate; primary, secondary, or tertiary amine (i.e., amino); amide; amidine; imine; azide; thiol, substituted or unsubstituted thioalkyl (e.g., thioether); isocyanate; isothiocyanate; phosphoryl; phosphate; phosphinate; sulfate; sulfonate; sulfamoyl; sulfonamide; sulfonyl; substituted or unsubstituted linear or branched alkyl, substituted or unsubstituted linear or branched alkenyl, substituted or unsubstituted linear or branched alkynyl, substituted or unsubstituted linear and branched alkoxy, substituted or unsubstituted C₃-C₁₀ cycloalkyl, cycloalkenyl, heterocyloalkyl, or heterocycloalkenyl, substituted or unsubstituted aryl or heteroaryl. One or more of R₁₃ can be present on the ring.

In some embodiments, Ar¹ is substituted with hydrogen, hydroxyl, nitro, amino, or azide; X is C═O; Y and Z are absent, and Ar³ is substituted with a halo group, a nitro group, or a combination of a halo and nitro group. In some embodiments, Ar¹ and Ar³ are phenyl rings substituted as described above.

In a preferred embodiment, Q is carbon, T is oxygen, and R is nitrogen.

In some embodiments, A and D are nitrogen.

In some embodiments, R₄ and R₁₃ are independently hydrogen or methyl.

In preferred embodiments, R₄ is methyl and R₁₃ is hydrogen.

In some embodiments, the aggregation promoters have the structure of formula IV:

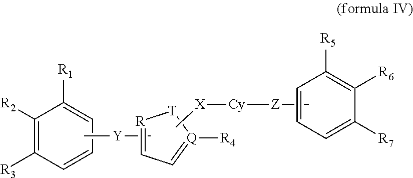

wherein X, Y, and Z are independently absent or selected from the group consisting of —C(═O)—, —S(═O)—, —C(═O)N(R₁₀), —N(R₁₁)—, —C(R₁₂)═C(R₁₃)—, and —C(R₁₄R₁₅)ₙ—;

wherein n is 0 to 10, preferably 0-6;

T, Q, and R are, as valence and stability permit, independently selected from C(R₈R₉), nitrogen, oxygen, phosphorous, silicon, sulfur, selenium, boron, and arsenic; and

Cy is a 4-7 membered substituted or unsubstituted cyclic or heterocyclic group;

wherein R₁-R₁₅ independently are absent, or are selected from hydrogen; halogen; hydroxy; nitro; nitrile; isonitrile; urea; guanidine; cyano; carbonyl, such as formyl, acyl, or carboxyl; thiocarbonyl, such as thioester, thioacetate, or thioformate; primary, secondary, or tertiary amine (i.e., amino); amide; amidine; imine; azide; thiol, substituted or unsubstituted thioalkyl (e.g., thioether); isocyanate; isothiocyanate; phosphoryl; phosphate; phosphinate; sulfate; sulfonate; sulfamoyl; sulfonamide; sulfonyl; substituted or unsubstituted linear or branched alkyl, substituted or unsubstituted linear or branched alkenyl, substituted or unsubstituted linear or branched alkynyl, substituted or unsubstituted linear and branched alkoxy, substituted or unsubstituted C₃-C₁₀ cycloalkyl, cycloalkenyl, heterocyloalkyl, or heterocycloalkenyl, substituted or unsubstituted aryl or heteroaryl.

In some embodiments, Cy is a substituted 5-7 membered unsaturated ring containing 2 nitrogen atoms, wherein one nitrogen atom is bonded to X and another nitrogen atom is bonded to Z.

In a preferred embodiment, Cy is a substituted piperazine, wherein one nitrogen is bonded to X and the second nitrogen is bonded to Z, Y and Z are absent, X is C═O, T is oxygen, Q is carbon, and R is nitrogen.

In some embodiments, R₁-R₃ and R₅-R₇ are selected from a halo group, a nitro group, or a combination of a halo and nitro group.

In preferred embodiments, R₄ is a methyl group.

In some embodiments, the aggregation promoters have the structure of formula V:

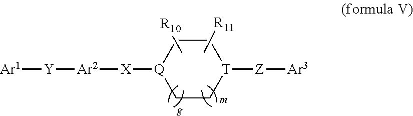

wherein Ar¹, Ar², and Ar³ are each independently substituted or unsubstituted aryl or heteroaryl groups

X, Y, and Z are independently absent or selected from the group consisting of —C(═O)—, —S(═O)—, —C(═O)N(R₁), —N(R₂)—, —C(R₃)═C(R₄)—, and —C(R₅R₆)ₙ—

n, g, and m are independently 0 to 10, preferably 0 to 6;

Q and T are independently selected from nitrogen or CR₇; and

R₁-R₇, R₁₀, and R₁₁ are independently selected from hydrogen; halogen; hydroxy; nitro; nitrile; isonitrile; urea; guanidine; cyano; carbonyl, such as formyl, acyl, or carboxyl; thiocarbonyl, such as thioester, thioacetate, or thioformate; primary, secondary, or tertiary amine (i.e., amino); amide; amidine; imine; azide; thiol, substituted or unsubstituted thioalkyl (e.g., thioether); isocyanate; isothiocyanate; phosphoryl; phosphate; phosphinate; sulfate; sulfonate; sulfamoyl; sulfonamide; sulfonyl; substituted or unsubstituted linear or branched alkyl, substituted or unsubstituted linear or branched alkenyl, substituted or unsubstituted linear or branched alkynyl, substituted or unsubstituted linear and branched alkoxy, substituted or unsubstituted C₃-C₁₀ cycloalkyl, cycloalkenyl, heterocyloalkyl, or heterocycloalkenyl, substituted or unsubstituted aryl or heteroaryl.

In some embodiments, Q and T are both nitrogen.

In some embodiments, R₁₀ is a methyl group and R₁₁ is hydrogen. In another embodiment, R₁₀ and R₁₁ are both hydrogen.

In some embodiments, Y and Z are absent and X is C═O.

In some embodiments, g and m are 1.

In a preferred embodiment, Ar¹ and Ar³ are a substituted phenyl, Ar² is a substituted isoxazole, Y and Z are absent, X is C═O, Q and T are nitrogen, g and m are 1, R₁₀ is methyl and R₁₁ is hydrogen.

In some embodiments, the aggregation promoters have the structure of formula VI:

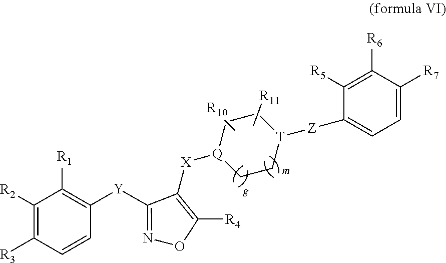

wherein X, Y, and Z are independently absent or selected from the group consisting of —C(═O)—, —S(═O)—, —SO₂—, —C(═O)N(R₁₂), —N(R₁₃)—, —C(R₁₄)═C(R₁₅)—, and —C(R₁₆R₁₇)ₙ—,

n, g, and m are independently 0 to 10, preferably 0 to 6;

Q and T are independently selected from nitrogen or CR₁₈; and

R₁-R₁₈ are independently selected from hydrogen, halo, hydroxyl, linear or branched C₁-C₁₀, preferably C₁-C₆ alkyl, linear or branched C₁-C₁₀, preferably C₁-C₁₀, preferably C₁-C₆ alkenyl, linear or branched C₁-C₁₀, preferably C₁-C₆ alkenyl, or linear and branched C₁-C₁₀, preferably C₁-C₆ alkoxy, amino, azide, cyano, nitro, nitrile, isonitrile, amide, carboxylate, urea, guanidine, isocyanate, isothiocyanate, and thioether.

In some embodiments, Q and T are both nitrogen.

In some embodiments, R₁₀ is a methyl group and R₁₁ is hydrogen. In other embodiments, both R₁₀ and R₁₁ are hydrogen.

In some embodiments, Y and Z are absent and X is C═O.

In some embodiments, g and m are 1.

In some embodiments, R₁-R₃ and R₅-R₇ are selected from a halo group, a nitro group, or a combination of a halo and nitro group.

In preferred embodiments, R₄ is a methyl group.

Some preferred compounds according to the invention are:
[4-(2-chloro-4-nitro-phenyl)-piperazinl-yl]-[3-(4-hydroxy-phenyl)-5-methylisoxazol-4-yl]-methanone;
[4-(2-chloro-4-nitro-phenyl)-piperazin-1-yl]-[3-phenyl-5-methyl-isoxazol-4-yl]-methanone;
[4-(2-chloro-4-nitro-phenyl)-piperazin-1-yl]-[3-(4-amino-phenyl)-methylisoxazol-4-yl]-methanone;
[4-(2-chloro-4-nitro-phenyl)-piperazin-1-yl]-[3-(4-azido-phenyl)-5-methylisoxazol-4-yl]-methanone;
[4-(2-chloro-4-nitro-phenyl)-piperazin-1-yl]-[3-(2-chloro-phenyl)-5-methylisoxazol-4-yl]-methanone;
[4-(2-chloro-4-nitro-phenyl)-2-methyl-piperain-1-yl]-[3-(2-chloro-phenyl)-5-methyl-isoxazol-4-yl]-methanone;
[4-(2-chloro-4-nitro-phenyl)-2-methyl-piperain-1-yl]-[3-phenyl-5-methylisoxazol-4-yl]-methanone;
[4-(4-nitro-phenyl)-piperazin-1-yl]-[3-(2-chloro-phenyl)-5-methyl-isoxazol-4-yl]-methanone;
and [4-(4-nitro-phenyl)-piperazin-1-yl]-[3-(2,6-dichloro-phenyl)-5-methyl-isoxazol-4-yl]-methanone.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are known in the art.

B. Formulations

Compounds which promote aggregation of nucleoprotein, and their pharmaceutically acceptable salts, can be formulated using standard techniques for enteral, parenteral, topical administration. Preferred compounds are those that belong to formulae I-VI. Effective dosages can be determined based on the in vitro assays known to those skilled in the art, such as the assays described in the examples. The compounds described herein can be formulated for enteral, parenteral, or topical administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients.

1. Parenteral Formulations

The compounds described herein can be formulated for parenteral administration. “Parenteral administration”, as used herein, means administration by any method other than through the digestive tract or non-invasive topical or regional routes. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

i. Controlled Release Formulations

The parenteral formulations described herein can be formulated for controlled release lease, including immediate release, delayed release, extended release, pulsatile release, and combinations thereof.

a.) Nano- and Microparticles

For parenteral administration, the one or more NP inhibitors, and optional one or more additional active agents, can be incorporated into microparticles, nanoparticles, or combinations thereof that provide controlled release. In embodiments wherein the formulations contains two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, the compounds and/or one or more additional active agents can be incorporated into polymeric microparticles which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material which is normally solid at room temperature and has a melting point of from about 30 to 300° C.

In some cases, it may be desirable to alter the rate of water penetration into the microparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins which are water insoluble, such as zein, can also be used as materials for the formation of drug containing microparticles. Additionally, proteins, polysaccharides and combinations thereof which are water soluble can be formulated with drug into microparticles and subsequently cross-linked to form an insoluble network. For example, cyclodextrins can be complexed with individual drug molecules and subsequently cross-linked.

Encapsulation or incorporation of drug into carrier materials to produce drug containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. Detailed descriptions of these processes can be found in “Remington—The science and practice of pharmacy”, 20th Edition, Jennaro et. al., (Phila, Lippencott, Williams, and Wilkens, 2000).

For some carrier materials it may be desirable to use a solvent evaporation technique to produce drug containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some embodiments drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin (Cortesi, R., et al., Biomaterials 19 (1998) 1641-1649). Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water soluble protein can be spray coated onto the microparticles and subsequently cross-linked by the one of the methods described above. Alternatively, drug containing microparticles can be microencapsulated within protein by coacervation-phase separation (for example, by the addition of salts) and subsequently cross-linked. Some suitable proteins for this purpose include gelatin, albumin, casein, and gluten. Polysaccharides can also be cross-linked to form a water-insoluble network. For many polysaccharides, this can be accomplished by reaction with calcium salts or multivalent cations which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

2. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition which may include plasticizers, pigments, colorants, stabilizing agents, and glidants. Delayed release dosage formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

i. Controlled Release Formulations

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In another embodiment, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

Extended Release Dosage Forms

The extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid) (anhydride), polymethacrylate, polyacrylamide, poly(methacrylic acid anhydride), and glycidyl methacrylate copolymers. In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename Eudragit®. In further preferred embodiments, the acrylic polymer comprises a mixture of two acrylic resin lacquers commercially available from Rohm Pharma under the tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. Edragit® S-100 and Eudragit® L-100 are also preferred. The code designations RL (high permeability) and RS (low permeability) refer to the permeability properties of these agents. Eudragit® RL/RS mixtures are insoluble in water and in digestive fluids. However, multiparticulate systems formed to include the same are swellable and permeable in aqueous solutions and digestive fluids. The polymers described above such as Eudragit® RL/RS may be mixed together in any desired ratio in order to ultimately obtain a sustained-release formulation having a desirable dissolution profile. Desirable sustained-release multiparticulate systems may be obtained, for instance, from 100% Eudragit® RL, 50% Eudragit® RL and 50% Eudragit® RS, and 10% Eudragit® RL and 90% Eudragit® RS. One skilled in the art will recognize that other acrylic polymers may also be used, such as, for example, Eudragit® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

3. Topical Formulations

Suitable dosage forms for topical administration include creams, ointments, salves, sprays, gels, lotions, emulsions, and transdermal patches. The formulation may be formulated for transmucosal, transepithelial, transendothelial, or transdermal administration. The compounds can also be formulated for intranasal delivery, pulmonary delivery, or inhalation. The compositions may further contain one or more chemical penetration enhancers, membrane permeability agents, membrane transport agents, emollients, surfactants, stabilizers, and combination thereof.

i. Topical Excipients

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

a.) Lotions, Creams, Gels, Ointments, Emulsions, and Foams

“Hydrophilic” as used herein refers to substances that have strongly polar groups that readily interact with water.

“Lipophilic” refers to compounds having an affinity for lipids.

“Amphiphilic” refers to a molecule combining hydrophilic and lipophilic (hydrophobic) properties

“Hydrophobic” as used herein refers to substances that lack an affinity for water; tending to repel and not absorb water as well as not dissolve in or mix with water.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) comprised of the drug dispersed or dissolved in a mixture of surfactant(s) and lipophilic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

The difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

4. Pulmonary Formulations

In one embodiment, the noscapine analogs are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorbtion occurs. The alveoli are covered by a thin epithelium without cilia or a mucus blanket and secrete surfactant phospholipids.

The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which then lead to the ultimate respiratory zone, the alveoli, or deep lung. The deep lung, or alveoli, are the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm3, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solutions is water, physiologically acceptable aqueous solutions containing salts and/or buffers, such as phosphate buffered saline (PBS), or any other aqueous solution acceptable for administration to a animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the noscapine analogs. An appropriate solvent should be used that dissolves the noscapine analogs or forms a suspension of the noscapine analogs. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the noscapine analogs in the lungs and that the excipients that are present are present in amount that do not adversely affect uptake of noscapine analogs in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, Calif.).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different EGS may be administered to target different regions of the lung in one administration.

Formulations for pulmonary delivery include unilamellar phospholipid vesicles, liposomes, or lipoprotein particles. Formulations and methods of making such formulations containing nucleic acid are well known to one of ordinary skill in the art. Liposomes are formed from commercially available phospholipids supplied by a variety of vendors including Avanti Polar Lipids, Inc. (Birmingham, Ala.). In one embodiment, the liposome can include a ligand molecule specific for a receptor on the surface of the target cell to direct the liposome to the target cell.

### IV. Methods of Treatment

Compounds that promote aggregation of proteins, glycoproteins, or protein-nucleic acid complexes may be used to prevent or reduce cellular growth, reduce or prevent the infectivity of viruses or bacteria, prevent or slow the development of resistance (a problem in anti-bacterial, anti-viral, and anti-cancer therapies), and to enhance the efficacy of traditional anti-proliferative therapies.

A. Disorders to be Treated

a.) Proliferative Disorders

Compounds that promote aggregation of proteins, glycoproteins, or protein-nucleic acid complexes are particularly useful for the treatment or prevention proliferative disorders, including cancer. In addition, the compounds can be used to prevent or treat disorders of abnormal cell proliferation generally, examples of which include, but are not limited to, types of cancers and proliferative disorders listed below.

Abnormal cellular proliferation, notably hyper-proliferation, can occur as a result of a wide variety of factors, including genetic mutation, infection, exposure to toxins, autoimmune disorders, and benign or malignant tumor induction.

There are a number of skin disorders associated with cellular hyper-proliferation including psoriasis, chronic eczema, atopic dermatitis, lichen planus, warts, pemphigus vulgaris, actinic keratosis, basal cell carcinoma and squamous cell carcinoma.

Other hyper-proliferative cell disorders include blood vessel proliferation disorders, fibrotic disorders, autoimmune disorders, graft-versus-host rejection, tumors and cancers.

Blood vessel proliferative disorders include angiogenic and vasculogenic disorders such as restenosis, retinopathies and atherosclerosis.

Fibrotic disorders include hepatic cirrhosis and mesangial proliferative cell disorders.

Mesangial disorders are brought about by abnormal proliferation of mesangial cells, and include various human renal diseases, such as glomerulonephritis, diabetic nephropathy, malignant nephrosclerosis, thrombotic micro-angiopathy syndromes, transplant rejection, and glomerulopathies.

Rheumatoid arthritis, Behcet's syndrome, acute respiratory distress syndrome (ARDS), ischemic heart disease, post-dialysis syndrome, leukemia, acquired immune deficiency syndrome, vasculitis, lipid histiocytosis, septic shock, and inflammation in general all at least partially involve hyperproliferation.

Specific types of cancers and diseases relating to cancer include Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphorria, Adult Lymphocytic Leukemia, Adult Non-Hodgkin's Lymphoma, Adult Primary Liver Cancer, Adult Soft Tissue Sarcoma, AIDS-Related Lymphorria, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphorria, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalanic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma. Endometrial Cancer, Ependymoma, Epithelial Cancer, Esophageal Cancer, Ewing's Sarcoma and Related Tumors, Exocrine Pancreatic Cancer, Extraeranial Germ Cell Tumor, Extragonadal Germ Cell Tumor, Extrahepatie Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lympho proliferative Disorders, Macroglobulinemia, Male Breast Cancer, Malignant Mesothelioma, Malignant Thymoma, Medulloblastomia, Melanoma, Mesothelioma, Metastatie Occult Primary Squamous Neck Cancer, Metastatic-Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyrigeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid, Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethial Cancer, Uterine Cancer, Uterine Sarcoma, Vaginal Cancer, Visual Pathway and Hypothalarruc Glioma, Vulvar Cancer, Waldenstroin's Macroglobulinemia, and Wilm's Tumor.

Viral infections, caused by both enveloped and non-enveloped viruses, including those that infect plants, animals, vertebrates, mammals and human patients can be prevented or treated with the compositions and methods described herein. The compounds and methods are suitable for treating all viruses that infect vertebrates, particularly humans, and particularly viruses that are pathogenic in animals and humans. The viral infections and associated and resultant diseases that can be treated include, but are not limited to CMV, RSV, arenavirus and HIV infections, and the diseases hepatitis, influenza, pneumonia, Lassa fever and AIDS. The International Committee on Taxonomy of Viruses contains a complete listing of viral strains, and is incorporated herein by reference.

In preferred embodiments, compounds identified in the screening methods are used as anti-proliferative agents. In another preferred embodiment, compounds that prevent nuclear accumulation of NP are used as anti-proliferative agents. In another embodiment, compounds that promote aggregation of nucleoproteins are used as anti-proliferative agents. In yet another embodiment, compounds that promote aggregation of proteins, glycoproteins, or protein-nucleic acid complexes are used as anti-proliferative agents.

In some embodiments, the diseases to prevent or treat include influenza A viral infections. Influenza A viruses that can be prevented or treated with formulations of the present method include H1N1, H2N2, H3N2, H5N1, H7N7, H1N2, H9N2, H7N2, H7N3, and H10N7. In preferred embodiments, the present formulations are useful for treatment of the influenza infection A strain caused by H1N1, H3N2, or H5N1.

Non-limiting examples of target bacteria that the present compounds and methods can be used to treat or prevent proliferation of are those that cause meningitis, including Steptococcus pneumoniae, Neisseria meningitides, Heaemophilus influenzae, Streptococcus agalactiea, and Listeria monocytogenes; those that cause otis media, including streptococcus pneumoniae; those that cause pneumonia, including streptococcus pneumoniae, haemophilus influenzae, Staphylococcus aureus, Mycoplasma pneumoniae, Chlamydia pneumoniae, Legionella pneumophila, and Mycobacterium tuberculosis; those which cause skin infections, including Staphylococcus aureus, Streptococcus pyogenes, and Pseudomonas aeruginosa; those which cause sexually transmitted diseases, including Chlamydia trachomatis, Neisseria gonorrhoeae, Treponema pallidum, Ureaplasma urealyticum, and Haemophilus ducreyi; those which cause eye infections, including Staphylococcus aureus, Neisseria gonorrhoeae, and Chlamydia trachomatis; those which cause sinusitis, including Streptococcus pneumoniae and Haemophilus influenzae; those which cause gastritis, including Helicobacter pylori; those which cause food poisoning, including Campylobacter jejuni, Salmonella, Shigella, Clostridium, Staphylococcus aureus, and Escherichia coli; and those which cause urinary tract infections, including Escherichia coli, Enterobacteriaceae, Stapholococcus saprophyticus, and Pseudomonas aeruginosa.

B. Dosages

The dosage of an anti-proliferative formulation necessary to prevent growth and proliferation depends upon a number of factors including the types of cell or virus that might be present, the environment into which the formulation is being introduced, and the time that the formulation is envisioned to remain in a given area.

Dosages preferably include compounds identified by the cell-free or cell-based screen, and are compounds that promote aggregation and/or inhibit nuclear nucleoprotein accumulation. Exemplary compounds belong to formulae I-VI.

The compounds can be administered to humans for the treatment of anti-proliferative diseases topically, orally, or parenterally. Typical doses for treatment are from about 0.1 to about 500 mg/kg, advantageously about 0.1 to 250 mg/kg/day given once or twice a day mg to 250 mg per day per kilogram of subject by body weight.

The compounds can be administered to humans for the treatment of influenza A infection by either the oral or parenteral routes and may be administered orally at dosage levels of about 0.1 to about 500 mg/kg, advantageously about 0.5 to 250 mg/kg/day given once or twice a day.

C. Mode of Administration

The formulations can be administered by any standard route, either systemically, topically or locally. Preferred routes of administration are by injection (parenterally) or orally using an enteric coating.

The formulations may be administered alone, but will generally be administered in admixture with a pharmaceutical carrier selected with regard to the intended route of administration and standard pharmaceutical practice. For example, they can be administered orally or in the form of tablets containing such excipients as starch or lactose, or in capsules either alone or in admixture with excipients, or in the form of elixirs or suspensions containing flavoring or coloring agents. In the case of humans, the compounds may be administered as syrup or enteric coated tablets. In addition, they can be injected parenterally, for example, intramuscularly, intravenously or subcutaneously. For parenteral administration, they are best used in the form of a sterile aqueous solution which can contain other solutes, for example, sufficient salt or glucose to make the solution isotonic.

In addition to the compound or compounds which promote protein aggregation, the compositions can contain one or more additional active agents. In one embodiment, the additional active agent is a compound that is used as a chemotherapeutic or anti-proliferative agent. Suitable compounds include, but are not limited to, 13-cis-Retinoic Acid, 2-Amino-6-2-CdA, 2-Chlorodeoxyadenosine, Mercaptopurine, 5-fluorouracil, 5-FU, 6-TG, 6-Thioguanine, 6-Mercaptopurine, 6-MP, Accutane Actinomycin-D, Adriamycin, Adrucil, Agrylin, Ala-Cort, Aldesleukin, Alemtuzumab, Alitretinoin, Alkaban-AQ, Alkeran, All-transretinoic, Alpha interferon, Altretamine acid, Amethopterin, Amifostine, Aminoglutethimide, Anagrelide, Anandron, Anastrozole, Arabinosylcytosine, Ara-C, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium, Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine, Cytosar-U, Cytoxan, liposomal Dacarbazine, Dactinomycin, Darbepoetin, Daunomycin, Daunorubicin, Daunorubicin, Daunorubicin, DaunoXome hydrochloride, liposomal Decadron, Delta-Cortef, Deltasone, Denileukin, diftitox, DepoCyt, Dexamethasone, Dexamethasone, dexamethasone sodium acetate phosphate, Dexasone, Dexrazoxane, DHAD, DIC, Diodex, Docetaxel, Doxil, Doxorubicin, Droxia, DTIC, DTIC-Dome, liposomal Duralone, Efudex, Eligard, Ellence, Eloxatin, Elspar, Emcyt, Epirubicin, Epoetin, Erbitux, Erwinia, Estramustine, L-asparaginase, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluoxymesterone, Flutamide, Folinic Acid, FUDR, Fulvestrant, G-CSF, Gefitinib, Gemcitabine, Gemtuzumab, Gemzar, Gleevec, Gliadel wafer, Glivec, GM-CSF, Goserelin, Halotestin, Herceptin, Hexadrol, Hexylen, Hexamethylmelamine, HMM, Hycamtin, Hydrea, Hydrocort Acetate, Hydrocortisone, Hydrocortisone, Hydroxyurea, Ibritumomab, Ibritumomab, Idamycin, Idarubicin, Tiuxetan, Ifex, IFN-alpha, Ifosfamide, IL-2, IL-11, Imatinib mesylate, Imidazole, Interferon alpha, Carboxamide, Interferon alpha-2b, Interleukin-2, Interleukin-11, Iressa, Irinotecan, Isotretinoin, Kidrolase, Lanacort, L-asparaginase, LCR, Letrozole, Leucovorin, Leukeran, Leukine, Leuprolide, Leurocristine, Leustatin, Lomustine, L-PAM, L-Sarcolysin, Lupron, Lupron Depot, Matulane, Maxidex, Mechlorethamine, Mechlorethamine, Medralone, Medrol, Megestrol, Melphalan, Mercaptopurine, Mesna, Mesnex, Methotrexate, Methylprednisolone, Meticorten, Mitomycin, Mitomycin-C, Mitoxantrone M-Prednisol, MTC, MTX, Mustargen, Mustine, Mutamycin, Myleran, Mylocel, Mylotarg, Navelbine, Neosar, Neulasta, Neumega, Neupogen, Nilandron, Nilutamide, Nitrogen Mustard, Novaldex, Novantrone, Octreotide, Oncospar, Oncovin, Ontak, Onxal, Oprevelkin, Orapred, Orasone, Oxaliplatin, Paclitaxel, Pamidronate, Panretin, Paraplatin, Pegaspargase, Pegfilgrastim, PEG-INTRON, PEG-L-asparaginase, Phenylalanine, Platinol, Platinol-AQ, Prednisolone, Prednisone, Prelone, Procarbazine, PROCRIT, Proleukin, Prolifeprospan 20, Purinethol, Raloxifene, Rheumatrex, Rituxan, Rituximab, Roveron-A, Rubex, Rubidomycin, Sandostatin, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vincasar, Vincristine, Vinorelbine, VLB, VM-26, VP-16, Vumon, Xeloda, Zanosar, Zevalin, Zinecard, Zoladex, Zoledronic acid, Zometa, and pharmaceutically acceptable salts thereof.

The compound or a pharmaceutically acceptable derivative or salts thereof can also be mixed with other active materials that do not impair the desired action, or with materials that supplement the desired action, including other anti-proliferation agents such as antibiotics, anti-fungals, anti-virals, or combinations thereof.

Variations in dosage and formulation will result based on the weight and condition of the subject being treated and the particular route of administration chosen as will be known to those skilled in the art

The present invention will be further understood by reference to following non-limiting examples.

### EXAMPLES

### Example 1

### In Vitro Evaluation of Nucleozin Binding Site Inhibitors

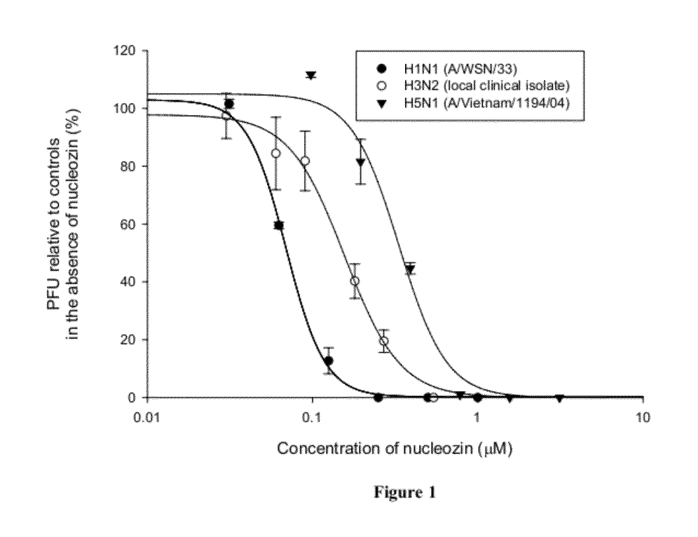

FIG. 1 shows a dose-response curve for nucleozin-treated mammalian cells infected with influenza A H1N1, H3N2, and H5N1 strains, graphing the percent plaque forming units (“PFU”) relative to controls in the absence of nucleozin as a function of the concentration of nucleozin (μM) for H1N1 (A/WSN/33) (filled circles), H3N2 (local clinical isolated) (open circles), and H5N1 (A/Vietnam/1194/04) (filled upside triangles).

### Example 2

### In Vivo Evaluation of a Nucleoprotein Aggregation Promoter

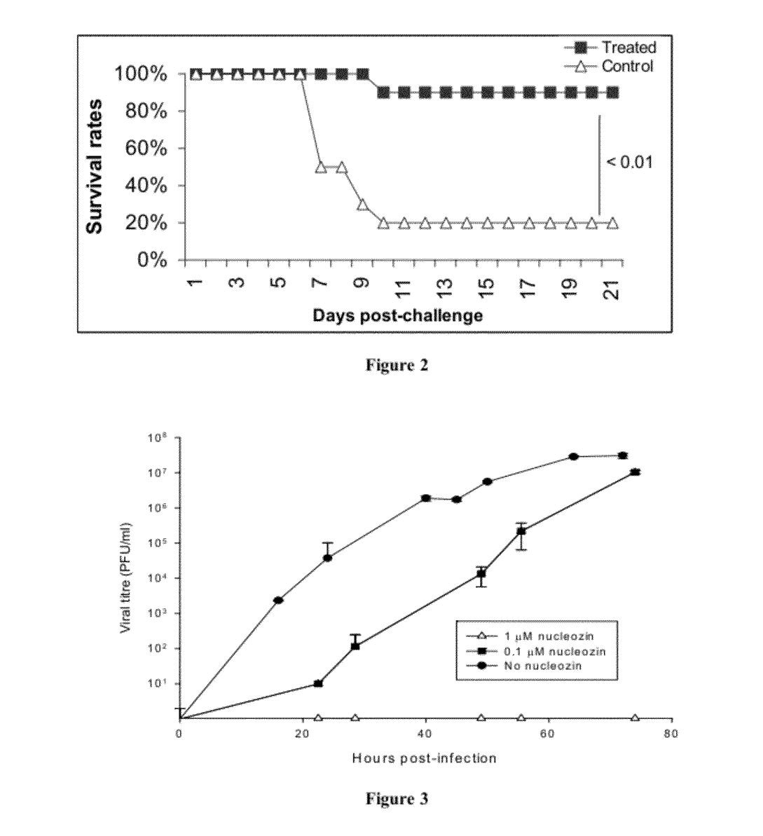

Five to seven week old BALB/c female mice in biosafety level 3 housing were used. The mice had access to standard pellet feed and water ad libitum. All experimental protocols followed the standard operating procedures of the approved biosafety level 3 animal facilities and were approved by the Animal Ethics Committee. One group (13 mice/group) of the mice was intraperitoneally (i.p.) injected with 100 μl of 2.5 mM of nucleozin (treated group) and the other group (13 mice) was injected with PBS (control group) one hour before inoculating the mice intranasally (i.n.) with 2×10⁴ TCID₅₀ of the A/Vietnam/1194/04 H5N1 virus in 20 μl 0.25 mM of the drug or PBS. The mice were given 100 μl of 2.5 mM nucleozin, administered twice a day i.p. or PBS for five days. Animal survival and general conditions were monitored for 21 days or till death. Statistical analysis of survival rate and viral load was performed by chi square test and the paired two-tailed Student's t test using Stata statistical software, respectively. Results were considered significant at P≦0.05. The results are shown in FIG. 2.

Discussion

Mice treated with nucleozin had a significantly higher survival rate after inoculation by influenza A virus H5N1 strain A/Vietnam/1194/04 than untreated controls. Without any treatment, 80% died after 10 days post inoculation. In the treated group, 90% of animals receiving two doses of nucleozin (250 nmole per dose) per day for 5 days survived for more than 21 days.

### Example 3

### Electrophoretic Mobility Shift Assay

An electrophoretic mobility shift assay was used to examine the effect of nucelozin on the RNA binding activity of the nucleoprotein. Purified recombinant wild type or Y289H variant nucleoprotein were incubated with nucleozin at room temperature for 30 min, then a 24-nucleotide RNA oligomer 16 was added and incubated for another 30 min. Nuclease-free water was added up to 10 μl. Final concentration of the small RNA oligomer was 2 μM and molar ratio of nucleoprotein:RNA was kept at 4:1. After incubation, the samples were mixed with 3 μl 6× DNA loading dye (0.25% bromophenol blue, 0.25% xylene xyanol, 40% surcose) and loaded into sample wells of non-denaturing 4-12% gradient Bis-Tris NuPAGE gel equilibrated by pre-electrophoresis at 50 V in 1×TBE. Samples were separated by electrophoresis at a constant voltage of 150 V for 35 min at room temperature in 1×TBE. The gel was first visualized by ethidium bromide staining for RNA shift patterns followed by staining with Coomassie brilliant blue G-250 for nucleoprotein shift patterns. For examining the effects of nucleozin-nucleoprotein interactions in vitro in the absence of RNA, we used native PAGE 4-16% Bis-Tris gradient gel for the separation of nucleoprotein under native conditions.

The aggregation-inducing agent nucleozin causes a dose-dependent reduction of nucleoprotein-RNA complex that runs into the 4-12% polyacrylamide gradient gel). The result suggested that nucleozin induces the formation of very large nucleoprotein-RNA aggregates that are too big to get into the gradient gel during electrophoresis. In the absence of RNA, the nucleozin treatment also reduces the amount of nucleoprotein running into the native gradient gel in a dose-dependent manner as judged by the intensities of Coomassie blue stained nucleoprotein in each lane, presumably due to formation of very large nucleoprotein aggregates.

### Example 4

### Cellular Immunofluorescence Microscopy

Detailed fluorescence microscopy studies using human alveolar basal epithelial (A549) cells as the host for influenza A/WSN/33 virus infection showed that an aggregation-inducing agent nucleozin is a potent antagonist of nucleoprotein accumulation in the nucleus, leading to a “halo” of dense nucleoprotein aggregates surrounding the perinuclear region in the cytoplasm at 3 hours post infection (FIG. 3). Because nucleoprotein failed to enter the nucleus in the presence of nucleozin, nucleoprotein trapped in the cytoplasm was seen scattered randomly in host cells at 24 hours after infection.

### Example 5

### Nucleozin Triggers Time Dependent Aggregation of Influenza Nucleoprotein

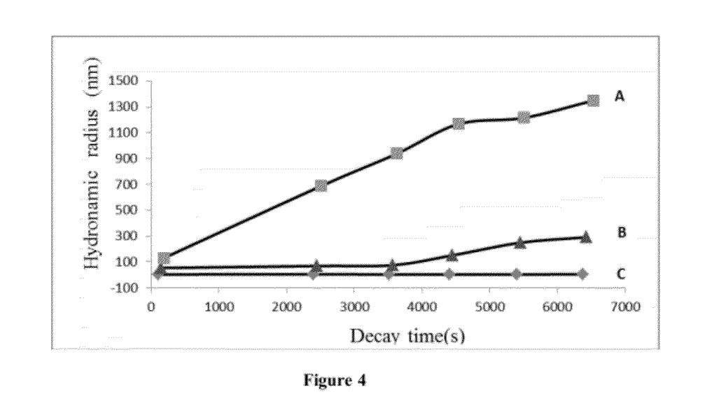

To characterize the kinetic of nucleoprotein (NP), the hydrodynamic radius of NP and it aggregation induced by nucleozin at 37° C. in 20 mM Tris-HCl buffer (pH 7.0) was evaluated using dynamic light scattering. As shown in FIG. 4, NP does not aggregate in the absence of nucleozin (Line A). However, upon the addition of nucleozin results in the aggregation of NP (Line B). Addition of RNA accelerates the aggregation of NP (Line C).

We claim:

1. A method for identifying compounds that promote aggregation comprising:
a.) treating cells with one or more test compounds for a period of time;
b.) adding a fixing solution to stop protein translation;
c.) treating the cells with a fluorescent antibody that binds specifically to a protein, glycoprotein, or protein-nucleic acid complex; and
d.) determining the presence or absence of aggregation,
wherein if aggregation is present in (d), the test compound is identified as a compound that may promote aggregation.

2. A cell-free method for identifying compounds that promote cytosolic nucleoprotein aggregation of proteins, glycoproteins, or protein-nucleic acid complexes comprising:
a.) combining bovine serum albumin and one or more proteins, glycoproteins, or protein-nucleic acid complexes;
b.) adding the mixture of (a) to a multi-well plate;
c.) transferring a test compound to one or more wells of the multi-well plate;
d.) incubating the plate of (c); and
e.) determining the presence of aggregation,
wherein if aggregation is present in (e), the test compound is identified as a compound that may promote aggregation.

3. The method of claim 1, wherein the assay is done in a multi-well format.

4. The method of claim 2, wherein the assay is done in a multi-well format.

5. The method of claim 2, wherein the target is a nucleoprotein.

6. The method of claim 1, wherein protein aggregation is determined by immunofluorescence microscopy, plate reader, or centrifugation.

7. The method of claim 2, wherein protein aggregation is determined by immunofluorescence microscopy, plate reader, or centrifugation.

8. The method of claim 1, wherein washes or aspirations are performed between steps.

9. The method of claim 2, wherein washes or aspirations are performed between steps.

10. A compound identified by the method of claim 1.

11. A compound identified by the method of claim 2.

12. A formulation comprising one or more compounds of claim 10.

13. A formulation comprising one or more compounds of claim 11.

14. The formulation of claim 12 further comprising additional agents selected from the group consisting of diluents, binders, lubricants, disintegrators, fillers, coating compositions, and combinations thereof.

15. The formulation of claim 13 further comprising additional agents selected from the group consisting of diluents, binders, lubricants, disintegrators, fillers, coating compositions, and combinations thereof.

16. A method for treating or preventing viral infection, bacterial infection, cancer, or a hyper-proliferative disease in a patient in need thereof comprising administering to a patient an effective amount of the formulation of claim 12.

17. The method of claim 16, wherein the formulation is administered topically, enterally, or parenterally.

18. The method of claim 16, wherein the influenza A infection is selected from the group consisting of H1N1, H3N2, and H5N1.

19. A method for treating or preventing viral infection, bacterial infection, cancer, or a hyper-proliferative disease in a patient in need thereof comprising administering to a patient an effective amount of the formulation of claim 13.

20. The method of claim 19, wherein the formulation is administered topically, enterally, or parenterally.

21. The method of claim 19, wherein the influenza A infection is selected from the group consisting of H1N1, H3N2, and H5N1.

22. A method for treating or preventing viral infection, bacterial infection, cancer, or a hyper-proliferative disease comprising administering to the patient an effective amount of a formulation of claim 12, wherein administration occurs before infection.

23. The method of claim 22, wherein the nucleoprotein is influenza A nucleoprotein.

24. The method of claim 22, wherein the formulation is administered topically, enterally, or parenterally.

25. The method of claim 22, wherein the influenza A infection is selected from the group consisting of H1N1, H3N2, and H5N1.

26. A method for treating or preventing viral infection, bacterial infection, cancer, or a hyper-proliferative disease comprising administering to the patient an effective amount of a formulation of claim 12, wherein administration occurs before infection.

27. A method for treating or preventing viral infection, bacterial infection, cancer, or a hyper-proliferative disease comprising administering to the patient an effective amount of a formulation of claim 13, wherein administration occurs before infection.

28. A method of treating or preventing viral infection, bacterial infection, cancer, or a hyper-proliferative disease in patient in need thereof comprising administering an effective amount of a compound that binds to a nucleoprotein binding site.

29. The method of claim 28, wherein the nucleoprotein is influenza A nucleoprotein.

30. The method of claim 28, wherein the formulation is administered topically, enterally, or parenterally.

31. The method of claim 25, wherein the influenza A infection is selected from the group consisting of H1N1, H3N2, and H5N1.

32. A method of treating or preventing viral infection, bacterial infection, cancer, or a hyper-proliferative disease in a patient in need thereof comprising administering an effective amount of a compound that inhibits nuclear accumulation of a nucleoprotein.

33. The method of claim 32, wherein the nucleoprotein is influenza A nucleoprotein.

34. The method of claim 32, wherein the formulation is administered topically, enterally, or parenterally.

35. The method of claim 33, wherein the influenza A infection is selected from the group consisting of H1N1, H3N2, and H5N1.

36. A method of treating or preventing viral infection, bacterial infection, cancer, or a hyper-proliferative disease in a patient in need thereof comprising administering an effective amount of a compound that promotes aggregation of a nucleoprotein.

37. The method of claim 36, wherein the formulation is administered topically, enterally, or parenterally.

38. The method of claim 36, wherein the influenza A infection is selected from the group consisting of H1N1, H3N2, and H5N1.

39. A method of treating or preventing influenza A infection in a patient in need thereof comprising administering an effective amount of a compound identified by the method of claim 1.

40. The method of claim 39, wherein the dosage is from about 0.1 mg to about 250 mg per day per kilogram of body weight.

41. A method of treating or preventing influenza A infection in a patient in need thereof comprising administering an effective amount of a compound identified by the method of claim 2.

42. The method of claim 41, wherein the dosage is from about 0.1 mg to about 250 mg per day per kilogram of body weight.

43. A method of treating or preventing influenza A infection in a patient in need thereof comprising administering an effective amount of a compound identified by the method of claim 1.

44. The method of claim 43, wherein the dosage is about 250 mg per day per kilogram of body weight.

45. The method of claim 43, wherein the formulation is administered topically, enterally, or parenterally.

46. The method of claim 43, wherein the influenza A infection is selected from the group consisting of H1N1, H3N2, and H5N1.

47. A method of treating or preventing influenza A infection in a patient in need thereof comprising administering an effective amount of a compound identified by the method of claim 2.

48. The method of claim 47, wherein the dosage is about 250 mg per day per kilogram of body weight.

49. The method of claim 47, wherein the formulation is administered topically, enterally, or parenterally.

50. The method of claim 47, wherein the influenza A infection is selected from the group consisting of H1N1, H3N2, and H5N1.
